(19) United States
(12) Patent Application Publication
(10) Pub. No.: US 2023/0160013 A1
(43) Pub. Date: May 25, 2023

(54) METHODS AND KITS FOR IDENTIFYING ADVANCED PATERNAL AGE RELATED EPIGENETIC DYSREGULATION

(71) Applicant: FERTILITY LAB SCIENCES, LLC, Lone Tree, CO (US)

(72) Inventors: MICHELLE M. TIGNANELLI, Littleton, CO (US); MANDY KATZ-JAFFE, Denver, CO (US)

(21) Appl. No.: 17/920,336

(22) PCT Filed: Apr. 21, 2021
(86) PCT No.: PCT/US21/28422

§ 371 Date: Oct. 20, 2022

Related U.S. Application Data
(60) Provisional application No. 63/013,090, filed on Apr. 21, 2020.

(51) Int. Cl. C12Q 1/6883

(52) U.S. Cl. CPC C12Q 1/6883; C12Q 2600/154

(57) ABSTRACT

Methods and kits for the identification and screening of epigenetic dysregulation in a blastocyst, sperm, or sperm population are provided. Identification and screening of epigenetic dysregulation is particularly tied to a number of candidate genes, termed autism spectrum disorder genes, schizophrenia genes, bipolar disorder genes, or opioid signaling pathway genes.

### RELATED APPLICATIONS

This application claims the benefit of U.S. Provisional application No. 63/013,090 titled “Methods and Kits for Identifying Advanced Paternal Age Related Epigenetic Dysregulation” filed Apr. 21, 2020 which is incorporated herein by reference.

### TECHNICAL FIELD

The present invention relates to an association of advanced paternal age and increased risk of epigenetic events during embryogenesis. The present invention also relates to the identification of a number of genes that act as markers for epigenetic screening associated with adverse outcomes during embryogenesis.

### BACKGROUND OF THE INVENTION

Advanced paternal age (APA) has been associated with adverse outcomes including birth defects and childhood neurodevelopmental diseases, like autism spectrum disorder, schizophrenia, and bipolar disorder. In spite of this, the average age of first time mothers and fathers in the United States is increasing, such that the average mean age of pregnancy for first time mothers has gone from about 25 years old to about 26 years old in the last 25 years. The same trend is seen in first time fathers, as the average age of men having a first child has risen by about 3 years since the 1980s. As such, it can be anticipated that the incidence of adverse outcomes associated with APA will continue to rise. The ability to identify risk factors and to develop diagnostic screens for individuals of APA is of need in the art, such that individuals who choose to start a family at an older age can limit their risk of having a newborn with an adverse outcome.

The present invention is directed toward overcoming one or more of the problems discussed above.

### SUMMARY OF THE INVENTION

Disclosed herein are methods and kits useful in the identification, screening and diagnosis of epigenetic dysregulation during embryogenesis, for example epigenetic dysregulation in a blastocyst.

Embodiments herein include, for example, methods for identifying a blastocyst, sperm, or sperm population with increased epigenetic dysregulation, and in particular, epigenetic dysregulation associated with adverse outcomes during embryogenesis. The method includes obtaining a blastocyst, sperm, or sperm population of interest, and identifying DNA methylation errors in the blastocyst. An increased risk of adverse outcome is correlated to epigenetic dysregulation in a blastocyst, sperm, or sperm population having global DNA with an overall hypomethylated shift, or correlated with a blastocyst having one or more, or two or more, autism spectrum disorder, schizophrenia, or bipolar disorder candidate genes being hypermethylated. In aspects of the case, the autism spectrum disorder candidate genes include: ABAT, ABCA7, ADSL, ANKRD11, CACNA1C, CACNA1H, CCDC88C, CDH11, CIC, CLSTN3, CNTNAP2, CSMD1, DDX3X, DHCR7, DPP6, EHMT1, FLT1, GLIS1, GNAS, GRIN1, GRM8, HDAC6, ITGB7, KCNQ2, NRP2, NSD1, P4HA2, PACS1, PIK3R2, PXDN, SGSH, SHANK2, SHANK3, SLC38A10, TET2, TT12, ZNF804A, and UBE3B. In some aspects of the case, the schizophrenia candidate genes include TCF3 and ZNF804A. In some aspects of the case, the bipolar disorder candidate genes include COMT, DRD4, GRIN1, MBP, PRKCZ, SHANK2, TRPM2, and ZNF804A. In some aspects of the case, opioid signaling pathway candidate genes include any of the neurodevelopmental disease candidate genes recited above. In some cases, the methods can be used to screen blastocysts, sperm, or sperm population for epigenetic dysregulation, where a blastocyst, sperm, or sperm population having epigenetic dysregulation as described above would not be used for an implantation procedure during in vitro fertilization, or in the case of the sperm, not used to fertilize an egg. In some cases, the methods can be used to screen blastocysts, sperm, or sperm population for epigenetic dysregulation, and blastocysts, sperm, or sperm population can be ranked to allow for the selection of the healthiest embryo for implantation or allow for the healthiest sperm to be used for fertilization.

In other embodiments, methods are provided for determining a blastocyst's epigenetic status. Methods include determining the level of methylation and gene expression in one or more, or two or more, autism spectrum disorder, schizophrenia, or bipolar disorder candidate genes in the blastocyst. In some aspects, the level of methylation and gene expression in the autism spectrum disorder, schizophrenia, or bipolar disorder genes are compared to a known control having a normal, or non-phenotypic, epigenetic status (in some cases, normal or non-phenotypic epigenetic status is associated with the methylation of the same one or more, or two or more, autism spectrum disorder genes found in sperm of younger fathers, typically less than 35 years old). As noted above, the autism spectrum disorder candidate genes can be any one of ABAT, ABCA7, ADSL, ANKRD11, CACNA1C, CACNA1H, CCDC88C, CDH11, CIC, CLSTN3, CNTNAP2, CSMD1, DDX3X, DHCR7, DPP6, EHMT1, FLT1, GLIS1, GNAS, GRIN1, GRM8, HDAC6, ITGB7, KCNQ2, NRP2, NSD1, P4HA2, PACS1, PIK3R2, PXDN, SGSH, SHANK2, SHANK3, SLC38A10, TET2, TT12, ZNF804A, or UBE3B. The schizophrenia candidate genes can be TCF3 and ZNF804A. The bipolar disorder candidate genes can be COMT, DRD4, GRIN1, MBP, PRKCZ, SHANK2, TRPM2, and ZNF804A. Opioid signaling pathway candidate genes can be any of the neurodevelopmental disease candidate genes recited above, for example, CACNA1H, GRIN1, and PRKCZ.

In alternative methods for determining a blastocyst's epigenetic status, the method includes determining the global DNA methylation of the blastocyst, where a hypomethylated or hypermethylated shift in global DNA methylation, as compared to a normal epigenetic status, is considered an abnormal epigenetic status. An abnormal epigenetic status is associated with a higher risk for the blastocyst to result in an adverse outcome as compared to a blastocyst having a normal epigenetic status.

Finally, embodiments herein include kits for testing the epigenetic status of one or more cells. In one aspect, the one or more cells are from a blastocyst, and the kit includes means for determining methylation level of the blastocyst, and means for determining gene expression in two or more target genes in the blastocyst. The two or more genes in the blastocyst can be autism spectrum disorder genes. In some kits, instructions including a standard range of values for a normal range of methylation and gene expression for autism spectrum disorder candidate genes is provided. Instructions can also include the methodology for conducting the methylation and gene expression testing.

### BRIEF DESCRIPTION OF THE FIGURES

FIG. 1A and FIG. 1B demonstrate global DNA methylation in young and APA sperm. FIG. 1A illustrates the distribution of the average Young and APA means for significant sperm CpGs visualized using boxplots. The bottom and top of each box represent the 1st and 3rd quantiles of the distributions, and the line between represents the median. The distributions are significantly different according to a Mann-Whitney test (*p=1.33×10-21).

FIG. 1B shows LINE1 bisulfite pyrosequencing. CpG sites on the x-axis are followed by the average percent methylation across all CpGs for Young (gray) and APA (black) individuals. Each line represents results for individual sperm samples (n=18 Young and n=18 APA sperm samples). Significant hypermethylation is observed in APA (70%) relative to Young (67%), *p<0.05.

FIGS. 2A through 2L provide plots of sperm methylation validation results. Each plot represents pyrosequencing results for selected validation genes at distinct CpG sites within a significant DMR: FIG. 2A—CACNA1H; FIG. 2B—COMT; FIG. 2C—DRD4; FIG. 2D—GRIN1; FIG. 2E—KCNQ1; FIG. 2F—PRKCZ; FIG. 2G—SHANK2; FIG. 2H—SHANK3; FIG. 2I—TCF3; FIG. 2J—TRPM2; FIG. 2K—CNTNAP2; FIG. 2L—MBP; FIG. 2M—ZNF804A. CpG sites on the x-axis are followed by the average percent methylation across all CpGs for young (gray) and APA (black) individuals. Each line represents results for individual sperm samples (n=18 young and n=18 APA sperm samples). Significant hypomethylation in APA relative to young is demonstrated in plots (A-J), and significant hypermethylation in APA relative to young is demonstrated in plots (K-M); *p≤0.01, **p≤0.001

FIG. 3 provides plots demonstrating Sperm DNA methylation change by paternal age. Linear regression models demonstrate a significant negative association between sperm DNA methylation and paternal age in APA fathers (≥50 years; n=18 APA sperm samples). Models were fitted using the lm( ) function in R with default arguments, and those with p≤0.05 were considered significant. R-squared, slope, and p-values are displayed above each plot, and shaded gray areas around each black regression line represent a 95% confidence interval.

FIG. 4A and FIG. 4B illustrate sperm and blastocyst DNA methylation validation results for CACNA1H and SHANK2. Bar plots show the average methylation changes within a significant DMR for two selected validation genes, CACNA1H (FIG. 1A) and SHANK2 (FIG. 1B), in sperm (n=12) and blastocyst (n=12) for Young (grey) and APA (black) fathers. Error bars represent one standard deviation. Significant hypomethylation is demonstrated for both sperm and blastocyst in CACNA1H and for sperm in SHANK2. *p≤0.05.

### DESCRIPTION

The study of epigenetic changes in the genome has resulted in advances in the understanding and treatment of a number of disease states. In particular, epigenetic modifications have now been identified as contributing factors for certain cancers, cognitive dysfunction, autoimmune diseases, and possible reproductive disorders. Of particular relevance herein, the inventors have identified a number of epigenetic events that occur during embryogenesis that correlate with a higher likelihood of an adverse outcome in a newborn, e.g., birth defect, autism spectrum disorder, etc. As disclosed herein, an “epigenetic event” is any process that alters gene activity without changing a DNA sequence. In one aspect, the epigenetic event is DNA methylation. Surprisingly, the inventors have found that these identified epigenetic events are more likely to occur when the father donor is of an APA.

Prior to the present disclosure, a causal mechanism underlying paternal age effect had not been elucidated, nor had the mode of inheritance been conclusively established. As described herein, modifiable and inheritable epigenetic information, such as DNA methylation, can be generationally transmitted. A series of epigenetic reprogramming events occur during gametogenesis and immediately after fertilization (Reik, Dean, & Walter, 2001). Erasure and gamete-specific establishment of methylation marks occur during primordial germ cell development. Directly after fertilization, the male pronucleus undergoes a rapid, active demethylation process, while the female pronucleus undergoes passive replication-dependent demethylation, prior to re-establishment in the developing embryo. Imprinted genes, and perhaps other developmentally critical regions, elude the embryonic portion of epigenetic reprogramming, enabling epigenetic generational inheritance (Kobayashi et al., 2012).

Disclosed herein, and substantiated by the data provided in the examples, is the finding that compromised DNA methylation profile in aged sperm results in incomplete reprogramming during spermatogenesis. For the first time, the inventors demonstrate a generational correlation in sperm and embryo of an altered human methylation landscape associated with advanced paternal age, contributing to nonequivalent efficiency of methylation re-establishment throughout the human blastocyst genome. Aberrant epigenetic reprogramming is significantly enriched at genes essential for neurological development in both APA sperm and blastocysts, and provides a mechanistic link between the paternal age effect and offspring neurodevelopmental disorders.

As disclosed herein, epigenetic events that correlate with a higher likelihood of an adverse outcome include: (1) a hypomethylated shift in global blastocyst DNA; (2) a higher likelihood that any one or more genes in the blastocyst shows both abnormal methylation and abnormal gene expression (increased or decreased); (3) hypermethylation in one or more, or at least two or more, three or more, four or more, etc. of the following genes in a blastocyst: ABAT, ABCA7, ADSL, ANKRD11, CACNA1C, CACNA1H, CCDC88C, CDH11, CIC, CLSTN3, CNTNAP2, CSMD1, DDX3X, DHCR7, DPP6, EHMT1, FLT1, GLIS1, GNAS, GRIN1, GRM8, HDAC6, ITGB7, KCNQ2, NRP2, NSD1, P4HA2, PACS1, PIK3R2, PXDN, SGSH, SHANK2, SHANK3, SLC38A10, TET2, TT12, ZNF804A, or UBE3B (termed the autism spectrum disorder candidate genes herein); TCF3 and ZNF804A (termed schizophrenia candidate genes herein); COMT, DRD4, GRIN1, MBP, PRKCZ, SHANK2, TRPM2, and ZNF804A (termed bipolar candidate genes herein); or (4) hypermethylation and decreased gene expression in any one or more autism spectrum disorder candidate gene, schizophrenia candidate gene, or bipolar disorder candidate gene in the blastocyst.

Where one of the above epigenetic events is identified in a blastocyst, it can be used to remove the blastocyst as a possible in vitro fertilization candidate. This is particularly true where the sperm donor for the blastocyst is of APA, or where the sperm donor experienced famine or some other stressful environmental or emotional disruption to epigenetic information. In other aspects, one or more blastocysts can be ranked by the degree or amount of epigenetic events such that the healthiest blastocyst can be chosen for implantation.

It is also disclosed herein that the autism spectrum disorder candidate genes, schizophrenia candidate genes, and/or bipolar disorder candidate genes can be used as markers in epigenetic testing, particularly, as markers of potential adverse events during embryogenesis. These genes are associated with altered methylation (either hyper or hypo) and altered expression (either increased or decreased) and are now shown (herein) associated with APA. These findings allow for the development of new screening strategies.

Embodiments herein provide methods and kits for the identification and/or screening of blastocysts for epigenetic events, and in particular, for epigenetic events tied to adverse outcomes during reproduction. Embodiments herein provide methods and kits for the identification and/or screening of sperm for epigenetic events, for example, epigenetic events tied to neurodevelopmental disorders in offspring.

Embodiments herein include methods for identifying a blastocyst with increased epigenetic dysregulation, and in particular, epigenetic dysregulation associated with adverse outcomes during embryogenesis, i.e. adverse outcomes resulting in offspring affected by neurodevelopmental disorders. The method includes, obtaining a blastocyst of interest and identifying DNA methylation errors in the blastocyst. An increased risk of epigenetic dysregulation is correlated with a blastocyst having global DNA with an overall hypomethylated shift, or correlated with a blastocyst having one or more, or two or more, autism spectrum disorder, schizophrenia, or bipolar disorder candidate genes being hypermethylated. In aspects of the case, the autism spectrum disorder candidate genes include ABAT, ABCA7, ADSL, ANKRD11, CACNA1C, CACNA1H, CCDC88C, CDH11, CIC, CLSTN3, CNTNAP2, CSMD1, DDX3X, DHCR7, DPP6, EHMT1, FLT1, GLIS1, GNAS, GRIN1, GRM8, HDAC6, ITGB7, KCNQ2, NRP2, NSD1, P4HA2, PACS1, PIK3R2, PXDN, SGSH, SHANK2, SHANK3, SLC38A10, TET2, TT12, ZNF804A, or UBE3B. The schizophrenia candidate genes include TCF3 and ZNF804A. The bipolar disorder candidate genes include COMT, DRD4, GRIN1, MBP, PRKCZ, SHANK2, TRPM2, and ZNF804A. Opioid signaling pathway candidate genes can be any of the neurodevelopmental disease candidate genes recited above, for example, CACNA1H, GRIN1, and PRKCZ.

In some cases, the methods can be used to screen blastocysts for epigenetic dysregulation, where a blastocyst having epigenetic dysregulation would not be used for an implantation procedure.

In other embodiments herein, methods are provided for determining a blastocyst's epigenetic status. Methods include determining the level of methylation and gene expression in one or more, or two or more, autism spectrum disorder, schizophrenia, or bipolar disorder candidate genes in the blastocyst. In some aspects, the level of methylation and gene expression in the one or more, or two or more, autism spectrum disorder, schizophrenia, or bipolar disorder genes are compared to a known control having a normal or non-phenotypic epigenetic status. As noted above, the autism spectrum disorder candidate genes can be any one of ABAT, ABCA7, ADSL, ANKRD11, CACNA1C, CACNA1H, CCDC88C, CDH11, CIC, CLSTN3, CNTNAP2, CSMD1, DDX3X, DHCR7, DPP6, EHMT1, FLT1, GLIS1, GNAS, GRIN1, GRM8, HDAC6, ITGB7, KCNQ2, NRP2, NSD1, P4HA2, PACS1, PIK3R2, PXDN, SGSH, SHANK2, SHANK3, SLC38A10, TET2, TT12, ZNF804A, or UBE3B. The schizophrenia candidate genes can be TCF3 and ZNF804A. The bipolar disorder candidate genes can be COMT, DRD4, GRIN1, MBP, PRKCZ, SHANK2, TRPM2, and ZNF804A. Opioid signaling pathway candidate genes can be any of the neurodevelopmental disease candidate genes recited above, for example, CACNA1H, GRIN1, and PRKCZ.

In alternative methods for determining a blastocyst's epigenetic status, the method includes determining the global DNA methylation of the blastocyst, where a hypomethylated or hypermethylated shift in the global DNA methylation, as compared to a normal epigenetic status, is considered an abnormal epigenetic status.

Finally, embodiments herein include kits for testing the epigenetic status of a blastocyst, which include means for determining methylation level of the blastocyst, and means for determining gene expression in two or more target genes in the blastocyst. The two or more genes in the blastocyst can be autism spectrum disorder genes, schizophrenia genes, or bipolar disorder genes. Standard control values and instructions on how to perform the testing can also be provided.

Embodiments herein include, methods for identifying sperm or a sperm donor having a predisposition to producing offspring with neurodevelopmental disorders. The method includes obtaining an ejaculate sample from the sperm donor, identifying the degree of DNA methylation errors in the sperm population, and comparing the degree of DNA methylation errors in the donor sperm population to the degree of DNA methylation errors in a control sample. If the degree of DNA methylation errors in the donor sperm population exceeds a threshold indicating a cumulative risk of epigenetic dysregulation, the donor sperm will not be used to fertilize eggs used for in vitro fertilization. In some aspects, the degree of epigenetic changes in a sperm or sperm population allows for a ranking of the sperm or sample to determine the healthiest sperm or sperm sample for use in in vitro fertilization. The cumulative risk is correlated with a sperm population having global DNA with an overall hypomethylated shift, or correlated with a sperm population having a threshold degree of DNA methylation errors in one or more, or two or more, autism spectrum disorder, schizophrenia, or bipolar disorder candidate genes being hypermethylated. In aspects of the case, the autism spectrum disorder candidate genes include ABAT, ABCA7, ADSL, ANKRD11, CACNA1C, CACNA1H, CCDC88C, CDH11, CIC, CLSTN3, CNTNAP2, CSMD1, DDX3X, DHCR7, DPP6, EHMT1, FLT1, GLIS1, GNAS, GRIN1, GRM8, HDAC6, ITGB7, KCNQ2, NRP2, NSD1, P4HA2, PACS1, PIK3R2, PXDN, SGSH, SHANK2, SHANK3, SLC38A10, TET2, TT12, ZNF804A, or UBE3B. The schizophrenia candidate genes can be TCF3 and ZNF804A. The bipolar disorder candidate genes can be COMT, DRD4, GRIN1, MBP, PRKCZ, SHANK2, TRPM2, and ZNF804A. Opioid signaling pathway candidate genes can be any of the neurodevelopmental disease candidate genes recited above, for example, CACNA1H, GRIN1, and PRKCZ.

In some cases, the methods can be used to identify sperm exhibiting epigenetic dysregulation, where a sperm having epigenetic dysregulation would not be used for an in vitro fertilization. In some aspects, the methods can be used to rank sperm or a sperm population to permit selection of the healthiest sperm for in vitro fertilization.

In other embodiments herein, methods are provided for determining the epigenetic status of an individual sperm, or epigenetic status of a sperm population obtained from a sperm donor. Methods include determining the level of methylation and gene expression in one or more, or two or more, autism spectrum disorder, schizophrenia, and/or bipolar disorder candidate genes in the sperm or sperm population. In some aspects, the level of methylation and gene expression in the one or more, or two or more, autism spectrum disorder schizophrenia, and/or bipolar disorder genes are compared to a known control having a normal or non-phenotypic epigenetic status. As noted above, the autism spectrum disorder candidate genes can be any one of ABAT, ABCA7, ADSL, ANKRD11, CACNA1C, CACNA1H, CCDC88C, CDH11, CIC, CLSTN3, CNTNAP2, CSMD1, DDX3X, DHCR7, DPP6, EHMT1, FLT1, GLIS1, GNAS, GRIN1, GRM8, HDAC6, ITGB7, KCNQ2, NRP2, NSD1, P4HA2, PACS1, PIK3R2, PXDN, SGSH, SHANK2, SHANK3, SLC38A10, TET2, TT12, ZNF804A, or UBE3B. The schizophrenia candidate genes can be TCF3 and ZNF804A. The bipolar disorder candidate genes can be COMT, DRD4, GRIN1, MBP, PRKCZ, SHANK2, TRPM2, and ZNF804A. Opioid signaling pathway candidate genes can be any of the neurodevelopmental disease candidate genes recited above, for example, CACNA1H, GRIN1, and PRKCZ.

In alternative methods for determining a sperm or sperm population's epigenetic status, the method includes determining the global DNA methylation of the sperm or sperm population, where a hypomethylated or hypermethylated shift in the global DNA methylation, as compared to a normal epigenetic status, is considered an abnormal epigenetic status.

Finally, embodiments herein include kits for testing the epigenetic status of a sperm or sperm population, which include means for determining methylation level of the sperm population, and means for determining gene expression in one or more, or two or more, target genes in the sperm. The genes in the sperm can be autism spectrum disorder genes, schizophrenia genes, and/or bipolar disorder genes. Standard control values and instructions on how to perform the testing can also be provided.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

### EXAMPLES

The following examples are provided for illustrative purposes only and are not intended to limit the scope of the invention. Statistical analysis included Student's t test, Fisher's exact test, Mann-Whitney U test, Benjamini-Hochberg method, with significance at p<0.05.

### Example 1: Methylation Plays A Role in Epigenetic Dysregulation of Blastocysts

Objective: APA has been associated with adverse outcomes including birth defects and childhood neurodevelopmental diseases, like autism spectrum disorder. Epigenetic dysregulation, such as inherited DNA methylation errors from aged fathers may lead to permanent altered gene expression in offspring, resulting in a clinical phenotype. The aim of this example was to investigate the methylome and subsequent transcriptome of human blastocysts in association with APA.

Materials and Methods: Cryopreserved, transferrable quality, human blastocysts were donated with IRB approval and patient consent. Normozoospermic aged fathers (APA; ≥50 years) were compared to normozoospermic younger fathers (Young; ≤35 years) with only donor oocytes to eliminate known female factors. Blastocyst DNA (n=12) was bisulfite converted and sequenced using Methyl-MaxiSeq (Zymo Research), while blastocyst RNA (n=12) underwent small cell number RNA-seq (Illumina). Results were analyzed in conjunction with pathway analysis (DAVID 6.8). Transcription validation (n=24) was performed using qPCR with REST 2009 and methylation validation (n=10) utilized the PyroMark Q24 Advanced system (Qiagen). Statistical analysis included Student's t-test, ANOVA in R, Fisher's Exact Test and Pair Wise Fixed Reallocation Randomization Test where appropriate, with significance at p<0.05.

Results: An overall shift towards hypomethylation was observed in APA blastocysts (34% APA vs. 40% Young; p<0.05), with 57,286 hypomethylated and 49,709 hypermethylated CpG sites (p<0.05). RNA sequencing revealed 751 genes with significantly decreased, and 195 genes with significantly increased expression (p<0.05). Combining the methylome and transcriptome datasets, 319 genes were significantly and directionally altered in both (p<0.05). Pathway analysis identified “disease mutation” as a highly significant key functional annotation category that includes intellectual disability, tumor suppressor genes, neurodegeneration, and autism spectrum disorder. To date, validation of methylation increase for the autism spectrum candidate genes SHANK2 (45% APA vs. 33% Young; p<0.05) and ANKRD11 (75% APA vs. 43% Young; p<0.05), and the observed decreased effects on their gene expression have been confirmed (p<0.05).

Conclusions: This novel study investigated the impact of APA on epigenetic events during embryogenesis. The results reveal an overall hypomethylated shift in blastocysts derived from aged fathers, resulting in significantly altered transcription. Interestingly, autism spectrum candidate genes were primarily hypermethylated with subsequent decreased gene expression, providing a mechanistic link between the advanced paternal age effect and childhood neurodevelopmental disorders.

### Example 2: Increase of Parental Age Significantly Exacerbated Hypomethylation

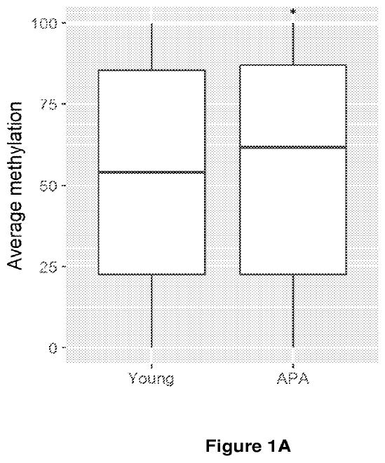

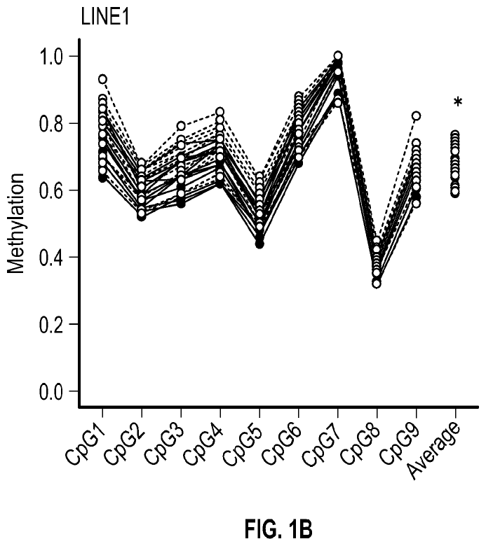

Six young (≤35 years) and six APA (≥50 years) normozoospermic sperm samples were used for global methylome analysis using a modified reduced representation bisulfite sequence (RRBS; Methyl Mini-Seq platform from Zymo Research) method, and an increase in global methylation was observed in the APA sperm epigenome and confirmed by LINE1 bisulfite pyrosequencing. See FIG. 1.

Sperm samples were prepared using two-layer PureSperm density-gradient centrifugation (Nidacon) followed by cell lysis to removed round cell contamination and storage in lysis buffer (Norgen Biotek). Surplus cryopreserved blastocysts were obtained from couples undergoing IVF treatment with informed consent and IRB approval. Similarly, six young and six APA blastocyst samples were analyzed using whole-genome bisulfite sequencing (WGBS; Methyl Maxi-Seq platform from Zymo Research). After identifying 49,722 and 106,995 CpGs statistically significant (p<0.05) between the young and APA groups for sperm and blastocysts, respectively, analysis resulted in 3,405 sperm DMRs and 3,997 blastocyst DMRs.

TABLE 1
APA Sperm and Blastocyst DMRs

| | APA sperm | | APA blastocyst | |
|---|---|---|---|---|
| | (n = 3,405 | | (n = 3,997 | |
| | DMRs) | | DMRs) | |
| | Paternal age: | | Paternal age: | |
| | Young (28.5 ± | | Young (32.7 ± | |
| | 2.1 years) | | 1.4 years) | |
| | APA (54.5 ± 3.9 | | APA (52.7 ± 3.0 | |
| | years) | | years) | |
| | Hypermethylated | Hypomethylated | Hypermethylated | Hypomethylated |
| Total DMRs | 1929 | 1476 | 1729 | 2268 |
| Significant | 8729 | 7262 | 6217 | 8244 |
| CpGs in DMRs | | | | |
| Average CpGs | 4.5 (range 3-35) | 4.9 (range 3-40) | 3.6 (range 3-25) | 3.6 (range 3-14) |
| per DMR | | | | |
| Average DMR | 211 bp (range 3- | 370 bp (range 3- | 550 bp (range 3- | 544 (range 4- |
| window width | 4069) | 3792) | 5432) | 4033) |
| Total associated | 612 | 810 | 1087 | 1457 |
| genes | | | | |
| Average genes | 0.32 | 0.55 | 0.63 | 0.64 |
| per DMR | | | | |
| Total unique | 568 | 766 | 803 | 1173 |
| associated genes | | | | |
| DMRs not | 1291 (67%) | 641 (43%) | 428 (25%) | 497 (22%) |
| associated with | | | | |
| a gene | | | | |

Hypomethylated DMRs in APA sperm were significantly enriched at CpG islands (p=1.80E-22), shelves (p=1.16E-175), and shores (p=1.76E-26). CpG island annotations were downloaded from the UCSC genome browser hg19. A similar trend was observed for hypomethylated DMRs in APA blastocysts.

Comparing sperm and blastocyst DMR-associated genes yielded a highly significant enrichment of genes between the two methylomes upon paternal aging (p=3.46E-55). Identification was made of 167 hypomethylated genes and 61 hypermethylated genes between the two methylomes with 10 genes exhibiting both hypo and hypermethylated DMRs.

TABLE 2
Overlapping DMRs

| | APA Sperm-Blastocyst Overlapping DMRs | | |
|---|---|---|---|
| | Hypermethylated | Hypomethylated | Any |
| Overlapping DMR- | 61 | 167 | 323 |
| Associated Genes | | | |
| Fold Enrichment | 2.36 | 4.31 | 3.19 |
| (odds ratio) | | | |
| OR 95% Confidence | 1.77-3.10 | 3.58-5.17 | 2.79-3.65 |
| Interval | | | |
| p-value | 2.25E−08 | 6.14E−45 | 3.46E−55 |

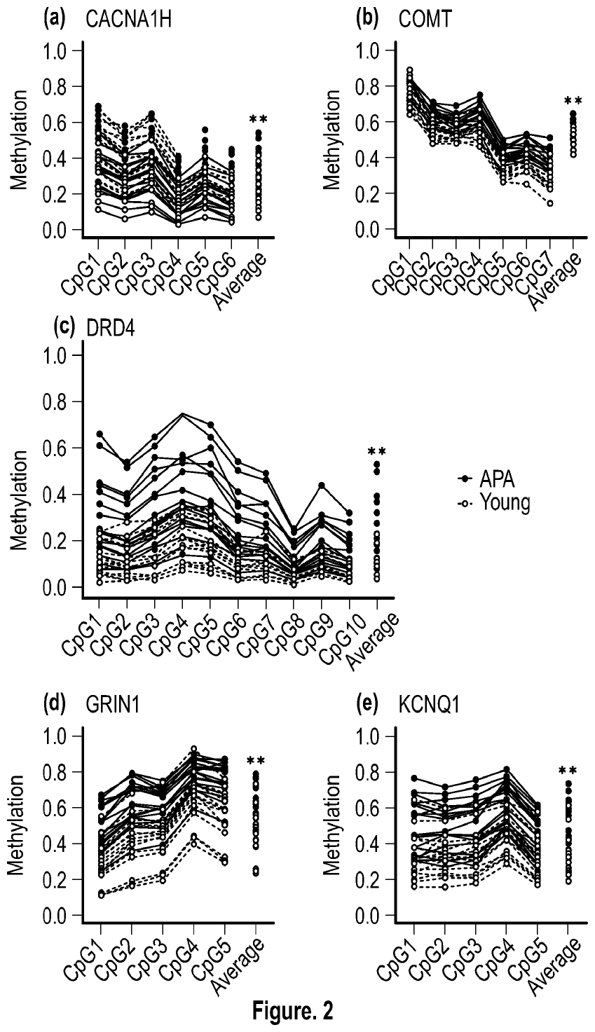

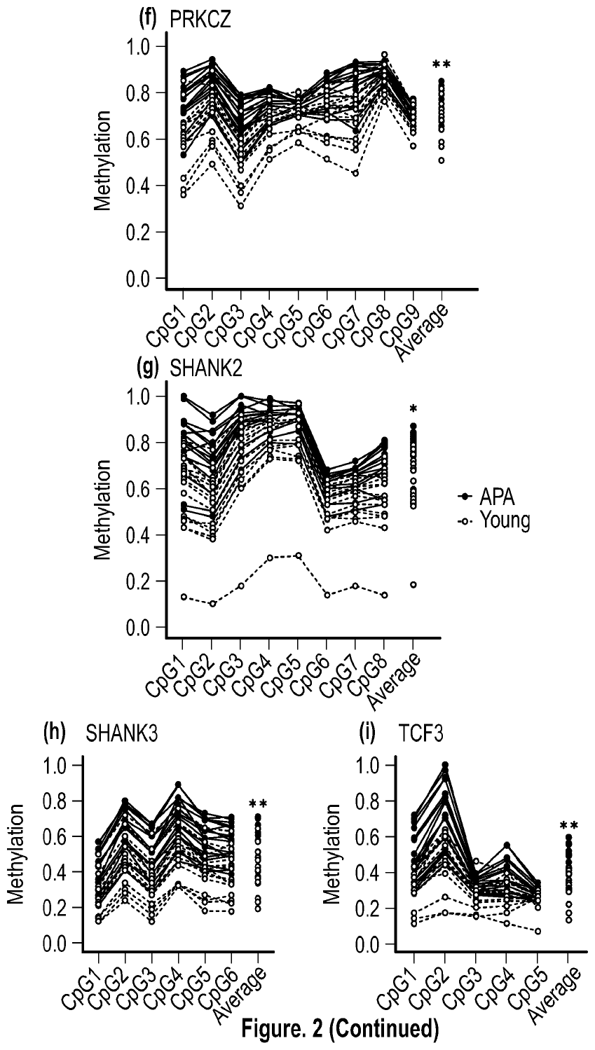

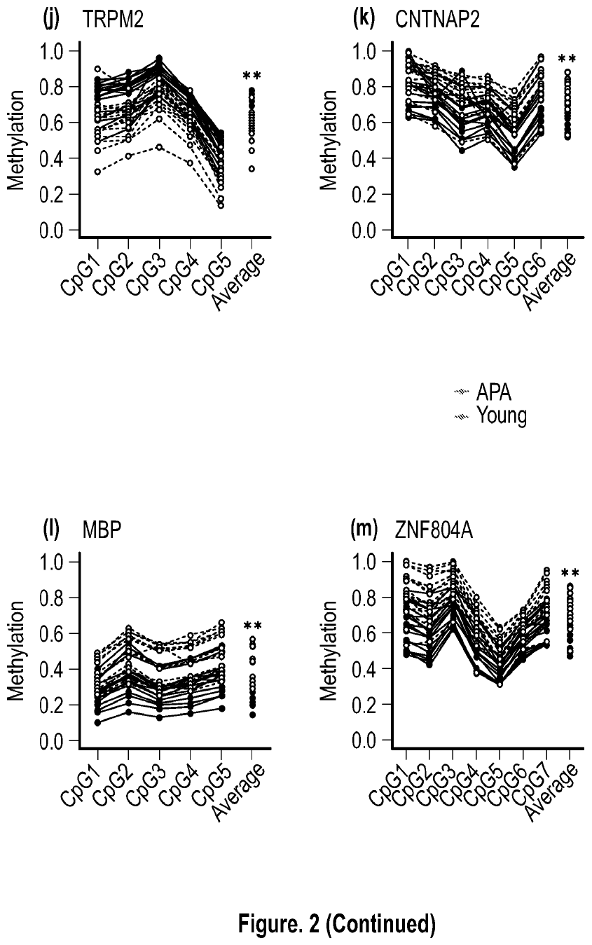

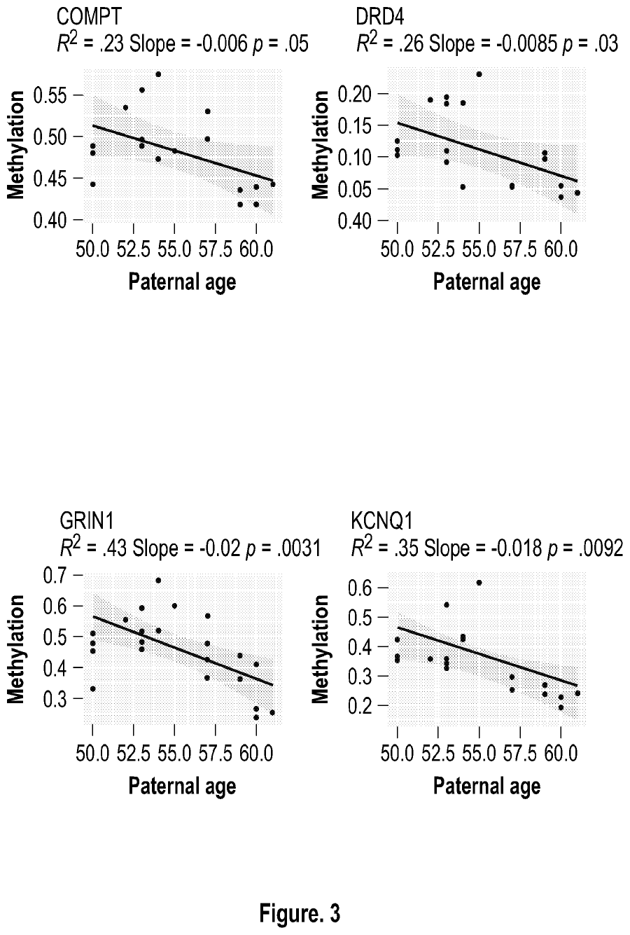

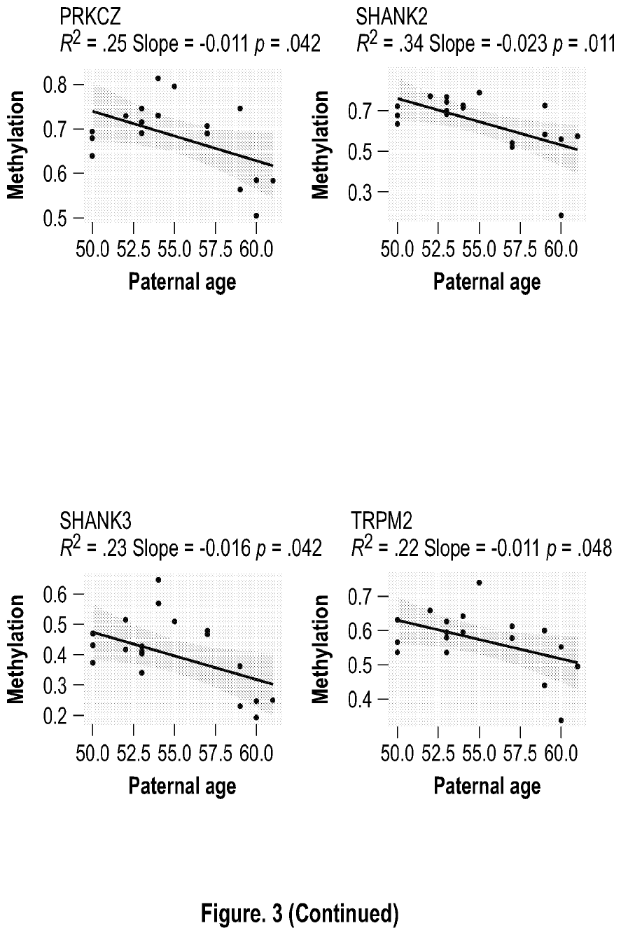

Normozoospermic sperm samples from an additional 12 young and 12 APA were used for methylation validation. See FIG. 2. Genes selected for methylation validation included altered DMRs, those identified in significant pathways, genes implicated in neurodevelopmental disorders, those localized to significant cytobands, imprinted genes, and those corresponding to published literature on aging sperm. See Table 3. Using linear regression models, hypomethylation was significantly exacerbated as paternal age increased from 50 years to over 60 years. See FIG. 3.

TABLE 3
DMR-associated genes previously identified in aging human sperm

| | Methylation | |
|---|---|---|
| Gene | Status | Association |
| AGRN | Hypomethylated | |
| ARC | Hypomethylated | |
| ATHL1 | Hypomethylated | |
| BEGAIN | Hypomethylated | |
| C7orf50 | Hypomethylated | |
| CACNA1H | Hypomethylated | Autism Spectrum Disorder, |
| | | Opioid Signaling |
| CCDC114 | Hypomethylated | |
| DAPK3 | Hypomethylated | |
| DLGAP2 | Hypomethylated | Imprinted |
| DRD4 | Hypomethylated | Bipolar Disorder |
| ELANE | Hypomethylated | |
| FOXK1 | Hypomethylated | |
| GET4 | Hypomethylated | Schizophrenia |
| GRIN1 | Hypomethylated | Autism Spectrum Disorder, |
| | | Bipolar Disorder, Opioid |
| | | Signaling |
| HOXA10 | Hypomethylated | |
| KCNA7 | Hypomethylated | |
| KCNF1 | Hypomethylated | |
| KCNQ1 | Hypomethylated | Imprinted |
| KDM2B | Hypomethylated | |
| LDLRAD4 | Hypomethylated | |
| LONP1 | Hypomethylated | |
| MPPED1 | Hypomethylated | |
| NADK | Hypomethylated | |
| NCOR2 | Hypomethylated | Schizophrenia, Frontal Cortex |
| | | of Autistic Brains |
| NSMF | Hypomethylated | |
| PALM | Hypomethylated | |
| PAX2 | Hypomethylated | |
| PITPNM1 | Hypomethylated | |
| PTPRN2 | Hypomethylated | Frontal Cortex of Autistic |
| | | Brains |
| PURA | Hypomethylated | |
| SECTM1 | Hypomethylated | |
| SLC22A18AS | Hypomethylated | Schizophrenia, Imprinted |
| SOHLH1 | Hypomethylated | |
| THBS3 | Hypomethylated | |
| UNKL | Hypomethylated | Schizophrenia |
| USP36 | Hypomethylated | |
| WFDC1 | Hypomethylated | |
| ZFPM1 | Hypomethylated | Schizophrenia |
| BCL11A | Hypermethylated | Autism Spectrum Disorder, |
| | | Bipolar Disorder |
| CCDC144NL | Hypermethylated | |
| FAM86C1 | Hypermethylated | |

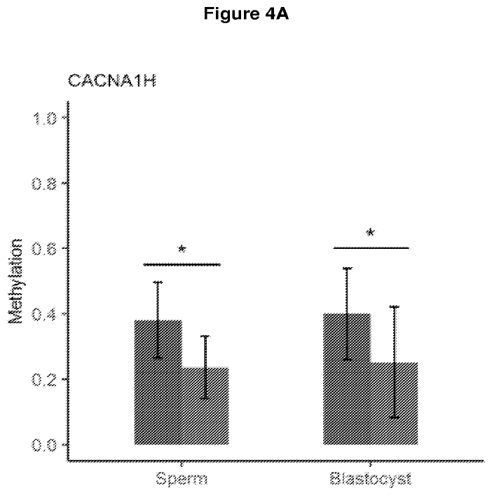

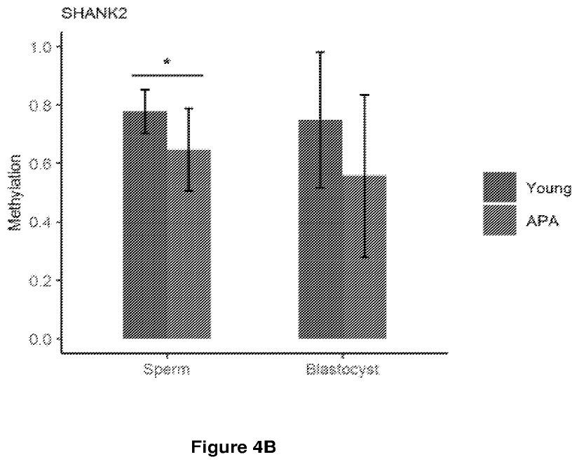

Twelve blastocysts (derived from young paternal age fathers) and 12 APA blastocysts were selected for blastocyst methylation validation in DMRs from two genes associated with neurodevelopmental disorders. Pyrosequencing data confirmed significant hypomethylation along the amplified CACNA1H DMR region (40% average methylation for young, 25% APA, p, 0.05) while validation of the SHANK2 blastocyst DMR trended towards hypomethylation (young 75%, APA 56%, p<0.1). See FIG. 4 and Table 4.

TABLE 4
Methylome read mapping statistics

| | | | | | | | | |
|---|---|---|---|---|---|---|---|---|
| | | | | Average | Bisulfite | Gene | | CpG |
| | Total Read | Mapping | Unique | CpG | Conversion | Body | Promoter | Island |
| Samples | Number | Efficiency | CpGs | Coverage | Rate | Coverage | Coverage | Coverage |

Sperm (Methyl-MiniSeq)

| | | | | | | | | |
|---|---|---|---|---|---|---|---|---|
| YNG 1 | 30,919,771 | 47% | 9,717,284 | 8X | 99% | 85% | 74% | 77% |
| YNG 2 | 33,860,516 | 46% | 9,864,863 | 8X | 99% | 85% | 74% | 79% |
| YNG 3 | 33,330,722 | 46% | 10,355,505 | 8X | 99% | 85% | 75% | 80% |
| YNG 4 | 32,212,578 | 42% | 10,145,132 | 7X | 99% | 85% | 74% | 78% |
| YNG 5 | 32,023,879 | 47% | 9,912,019 | 8X | 99% | 85% | 74% | 78% |
| YNG 6 | 32,236,741 | 48% | 9,837,071 | 8X | 99% | 85% | 75% | 79% |
| APA 1 | 31,247,009 | 47% | 10,129,874 | 8X | 99% | 85% | 75% | 79% |
| APA 2 | 32,567,449 | 45% | 10,220,222 | 8X | 99% | 85% | 75% | 79% |
| APA 3 | 32,471,117 | 42% | 9,873,598 | 7X | 99% | 85% | 74% | 77% |
| APA 4 | 30,202,170 | 43% | 9,663,748 | 7X | 99% | 85% | 73% | 77% |
| APA 5 | 31,611,911 | 45% | 9,816,449 | 8X | 99% | 85% | 74% | 77% |
| APA 6 | 35,743,721 | 48% | 10,036,958 | 9X | 99% | 85% | 76% | 81% |

Blastocysts (Methyl-MaxiSeq)

| | | | | | | | | |
|---|---|---|---|---|---|---|---|---|
| YNG 1 | 422,906,276 | 62% | 22,896,016 | 30X | 99% | 85% | 69% | 56% |
| YNG 2 | 484,339,064 | 61% | 28,370,405 | 30X | 99% | 86% | 71% | 60% |
| YNG 3 | 462,453,232 | 61% | 43,593,680 | 17X | 99% | 89% | 84% | 75% |
| YNG 4 | 481,752,559 | 64% | 29,478,372 | 31X | 98% | 87% | 76% | 67% |
| YNG 5 | 487,212,124 | 62% | 36,443,768 | 23X | 99% | 88% | 81% | 71% |
| YNG 6 | 489,702,951 | 63% | 36,094,786 | 24X | 98% | 88% | 77% | 68% |
| APA 1 | 502,958,355 | 61% | 42,018,862 | 21X | 99% | 89% | 83% | 78% |
| APA 2 | 507,076,029 | 63% | 22,090,645 | 40X | 99% | 86% | 71% | 58% |
| APA 3 | 510,044,505 | 62% | 38,726,517 | 23X | 98% | 88% | 76% | 66% |
| APA 4 | 466,725,178 | 61% | 38,469,550 | 20X | 98% | 89% | 81% | 70% |
| APA 5 | 448,954,497 | 60% | 38,299,371 | 19X | 99% | 88% | 80% | 68% |
| APA 6 | 495,812,948 | 63% | 37,621,620 | 25X | 99% | 87% | 74% | 64% |

### Example 3: Specific Chromosomal Regions are More Susceptible to Age-Related Methylation Alterations

Chromosomal enrichment for DMR-associated gene density at individual cytobands was analyzed to determine if specific chromosomal regions are more susceptible to age-related methylation alterations. Methylation alterations were not randomly distributed across the genome, but appear clustered at certain chromosomal locations with chromosome 19 having the greatest significance between young and APA samples (sperm p=5.51E-7, blastocyst p=9.01E-13, overlapping p=7.28E-6). Significant enrichment was identified at 5 cytobands in APA sperm, with four of the five independently enriched in APA blastocyst methylome, and 3 significant in the overlapping APA sperm and blastocyst gene list. See Table 5.

TABLE 5
Statistically significant cytobands

| | APA | | | Odds |
|---|---|---|---|---|
| Cytoband | Sample | P-value | FDR | Ratio |
| Chr10q26.3 | Sperm | 1.79E−04 | 2.91E−02 | |
| | Blastocyst | 3.93E−05 | 2.66E−03 | |
| | Overlapping | 2.25E−02 | N.S. | 5.29 |
| Chr11p15.5 | Sperm | 6.95E−08 | 2.45E−05 | |
| | Blastocyst | 4.92E−02 | N.S. | |
| | Overlapping | 2.98E−04 | 3.03E−02 | 5.95 |
| Chr16p13.3 | Sperm | 9.02E−08 | 2.45E−05 | |
| | Blastocyst | 2.43E−07 | 3.81E−05 | |
| | Overlapping | 6.50E−05 | 1.05E−02 | 4.20 |
| Chr17q25.3 | Sperm | 7.52E−05 | 1.53E−02 | |
| | Blastocyst | 2.81E−07 | 3.81E−05 | |
| | Overlapping | 6.59E−03 | N.S. | 3.80 |
| Chr19p13.3 | Sperm | 4.32E−19 | 3.51E−16 | |
| | Blastocyst | 3.76E−22 | 3.06E−19 | |
| | Overlapping | 5.62E−16 | 4.57E−13 | 9.89 |

Greatest enrichment for all three datasets was chr19p13.3 (sperm p=4.32E-19, blastocyst p=3.76E-22, overlapping p=5.62E-16). Analyzation of the colocalization of DMR-associated genes with known regions of nucleosome retention in sperm identified statistically significant enrichment with mononucleosomes for APA sperm DMRs (p=9.36E-29) and with presence of repressive histone mark H3K27me3 (p=7.21E-6). This colocalization with mononucleosomes was enhanced for the directionally overlapping DMRs identified in both sperm and blastocyst datasets (p=2.52E-18). See Table 6.

TABLE 6
Co-localization of DMR-associated genes with
histones and histone modifications

| | | | | |
|---|---|---|---|---|
| | | Fold | OR 95% | |
| | Overlapping | enrichment | confidence | |
| Histone gene set* | genes | (odds ratio) | interval | P-value |

APA Sperm DMRs

| | | | | |
|---|---|---|---|---|
| Mononucleosomes | 619 | 1.89 | 1.69-2.11 | 9.36E−29 |
| H3K27me3 | 275 | 1.38 | 1.20-1.59 | 7.21E−06 |
| H3K4me3 | 541 | 0.94 | 0.84-1.05 | N.S. |

APA Blastocyst DMRs

| | | | | |
|---|---|---|---|---|
| Mononucleosomes | 1074 | 2.19 | 2.00-2.39 | 1.50E−66 |
| H3K27me3 | 364 | 1.05 | 0.93-1.18 | N.S. |
| H3K4me3 | 957 | 1.08 | 0.98-1.18 | N.S. |

APA Sperm-Blastocyst Directional Overlapping DMRs

| | | | | |
|---|---|---|---|---|
| Mononucleosomes | 130 | 3.32 | 2.51-4.41 | 2.52E−18 |
| H3K27me3 | 31 | 0.90 | 0.59-1.32 | N.S. |
| H3K4me3 | 79 | 0.82 | 0.62-1.10 | N.S. |

*Hammoud, S. S. et al. Distinctive chromatin in human sperm packages genes for embryo development. Nature 460, 473-478, doi:10.1038/nature08162 (2009).

### Example 4: Effect of Paternal Aging Altering Methylation at Genes Linked to Neurodevelopmental Disorders

Neurological signaling pathways were found to be represented among genome regions that were differentially methylated in APA samples. The opioid signaling pathway was the top pathway for the directionally overlapping genes found in both APA sperm and blastocyst datasets. Genes successfully validated from the opioid signaling pathway include CACNA1H, GRIN1 and PRKCZ in APA sperm and CACNA1H in APA blastocysts. The pathway involving nNOS signaling in neurons was highlighted as the top canonical pathway impacted in the APA sperm methylome (p=2.08×10−4), which also comprises similar successfully validated genes (GRIN1 and PRKCZ). Additional pathways impacted by advanced paternal age can be found in Table 7.

TABLE 7
Top Canonical Pathways

| | | |
|---|---|---|
| Rank | Pathway | p-value |

APA sperm DMRs

| | | |
|---|---|---|
| 1 | nNOS signaling in neurons | 2.08E−04 |
| 2 | Sumoylation pathway | 2.50E−04 |
| 3 | Glutamate receptor signaling | 2.54E−04 |
| 4 | Opioid signaling pathway | 1.24E−03 |
| 5 | Dopamine-DARPP32 feedback in cAMP | 4.17E−03 |
| | signaling | |

APA blastocyst DMRs

| | | |
|---|---|---|
| 1 | Opioid signaling pathway | 2.45E−07 |
| 2 | Netrin signaling | 3.58E−07 |
| 3 | GPCR-mediated nutrient sensing in | 1.69E−06 |
| | enteroendocrine cells | |
| 4 | CREB signaling in neurons | 4.28E−06 |
| 5 | PPARα/RXRα Activation | 5.77E−06 |

APA sperm-blastocyst directional overlapping DMRs

| | | |
|---|---|---|
| 1 | Opioid signaling pathway | 2.14E−03 |
| 2 | Tight junction signaling | 4.84E−03 |
| 3 | Wnt/β-catenin signaling | 5.42E−03 |
| 4 | Role of Wnt/GSK-3β signaling in the | 5.71E−03 |
| | pathogenesis of influenza | |
| 5 | Phosphatidylethanolamine biosynthesis III | 9.26E−03 |

Publicly available gene lists were compared to the DMRs, and highly significant enrichment for genes was identified in three neurological disorders, specifically, autism spectrum disorder (p=1.94E-4), schizophrenia (p=5.55E-4), and bipolar disorder (p=4.73E-5). This enrichment was identified independently in the APA sperm DMRs and APA blastocyst DMRs. See Table 8. Genes were validated in APA sperm that are associated with autism spectrum disorder (CACNA1H, CNTNAP2, GRIN1, SHANK2, SHANK3, ZNF804A), schizophrenia (TCF3, ZNF804A), bipolar disorder (COMT, DRD4, GRIN1, MBP, PRKCZ, SHANK2, TRPM2, ZNF804A)) and in APA blastocysts (CACNA1H). The opioid signaling pathway is shared by all three candidate neurodevelopmental gene lists. See Table 9.

TABLE 8
Neurodevelopmental Disorder Associations

| | | | | |
|---|---|---|---|---|
| | | Fold | OR 95% | |
| | Overlapping | Enrichment | Confidence | |
| Disease | Genes | (odds ratio) | Interval | p-value |

APA Sperm DMRs

| | | | | |
|---|---|---|---|---|
| Autism spectrum | 35 | 2.48 | 1.68-3.57 | 7.76E−06 |
| disorder | | | | |
| Schizophrenia | 19 | 2.31 | 1.35-3.77 | 1.57E−03 |
| Bipolar Disorder | 63 | 1.93 | 1.45-2.53 | 8.24E−06 |

APA Blastocyst DMRs

| | | | | |
|---|---|---|---|---|
| Autism spectrum | 60 | 2.80 | 2.06-3.76 | 2.88E−10 |
| disorder | | | | |
| Schizophrenia | 40 | 3.40 | 2.30-4.94 | 2.42E−09 |
| Bipolar Disorder | 91 | 1.71 | 1.35-2.16 | 1.17E−05 |

APA Sperm-Blastocyst Directional Overlapping DMRs

| | | | | |
|---|---|---|---|---|
| Autism spectrum | 10 | 4.34 | 2.03-8.28 | 1.94E−04 |
| disorder | | | | |
| Schizophrenia | 7 | 5.30 | 2.07-11.38 | 5.55E−04 |
| Bipolar Disorder | 17 | 3.30 | 1.87-5.47 | 4.73E−05 |

TABLE 9
Top canonical pathways for neurode velopmental disorders

| Autism Spectrum | | | | | |
|---|---|---|---|---|---|
| Disorder | P-value | Schizophrenia | P-value | Bipolar Disorder | P-value |
| GABA Receptor | 2.19E−08 | Synaptic Long | 2.29E−06 | Serotonin Receptor | 1.26E−23 |
| Signaling | | Term | | Signaling | |
| | | Depression | | | |
| CREB Signaling in | 4.90E−07 | Synaptic Long | 6.03E−06 | Circadian Rhythm | 2.51E−23 |
| Neurons | | Term | | Signaling | |
| | | Potentiation | | | |
| Glutamate Receptor | 5.62E−06 | Dopamine- | 1.86E−05 | CREB Signaling in | 2.51E−19 |
| Signaling | | DARPP32 | | Neurons | |
| | | Feedback in | | | |
| | | cAMP Signaling | | | |
| Calcium Signaling | 4.68E−05 | p70S6K | 7.76E−05 | G-Protein Coupled | 6.31E−18 |
| | | Signaling | | Receptor Signaling | |
| Opioid Signaling | 5.25E−05 | CREB Signaling | 9.12E−05 | GABA Receptor | 3.98E−17 |
| Pathway | | in Neurons | | Signaling | |
| Amyotrophic Lateral | 6.46E−05 | Calcium | 1.95E−04 | Dopamine | 5.01E−17 |
| Sclerosis Signaling | | Signaling | | Receptor Signaling | |
| GPCR-Mediated | 6.76E−05 | Opioid | 1.05E−03 | cAMP-mediated | 3.16E−15 |
| Nutrient Sensing in | | Signaling | | signaling | |
| Enteroendocrine | | Pathway | | | |
| Cells | | | | | |
| nNOS Signaling in | 1.32E−04 | D-myo-inositol- | 1.23E−03 | Opioid Signaling | 3.98E−15 |
| Skeletal Muscle | | 5-phosphate | | Pathway | |
| Cells | | Metabolism | | | |
| Corticotropin | 3.02E−04 | 14-3-3-mediated | 1.38E−03 | Calcium Signaling | 6.31E−15 |
| Releasing Hormone | | Signaling | | | |
| Signaling | | | | | |
| Synaptic Long Term | 3.72E−04 | Role of NFAT in | 1.48E−03 | Neuroinflammation | 3.16E−14 |
| Depression | | Cardiac | | Signaling Pathway | |
| | | Hypertrophy | | | |

Using publicly available methylation profiling arrays, two brain tissues from autistic and nonautistic samples were analyzed; 141 (autistic) and 62 (nonautistic) DMRs were identified. The genes within these DMRs significantly overlapped APA sperm (frontal cortex p=5.78E-9, cingulate cortex p=1.62E-4) and APA blastocyst (frontal cortex p=1.94E-16, cingulate cortex p=3.24E-5) DMR-associated gene lists. The frontal cortex was determined to be significantly enriched for opioid signaling (p=2.14E-3) using pathway analysis of the genes associated with the DMRs. See Table 10.

TABLE 10
Pathway analysis for DMR-associated genes in autistic brains
compared with APA sperm and APA blastocyst

| Canonical Pathway | Autistic Frontal | APA | APA |
|---|---|---|---|
| (p ≤ 0.01) | Cortex | Sperm | Blastocyst |
| Opioid Signaling | 2.14E−03 | 1.23E−03 | 2.45E−07 |
| Pathway | | | |
| Melatonin Signaling | 2.24E−03 | 6.17E−03 | 3.72E−04 |
| Dopamine-DARPP32 | 2.88E−03 | 4.17E−03 | 1.10E−05 |
| Feedback in cAMP | | | |
| Signaling | | | |
| Neuregulin Signaling | 5.13E−03 | 5.25E−03 | N.S. |

The APA datasets were further compared to the current list of known and putative human-imprinted genes. APA sperm had 19 hypomethylated and 7 hypermethylated significant DMRs associated with imprinted genes. APA blastocysts had 22 hypomethylated and 10 hypermethylated significant DMRs associated with imprinted genes, with 6 imprinted genes overlapped in both datasets (p<0.05). See Table 11.

TABLE 11
DMR-associated imprinted genes

| APA | | | | Imprinting | Expressed |
|---|---|---|---|---|---|
| Sample | DMR | Chr | Imprinted Gene | Status | Allele |
| Sperm | Hypomethylated | chr1 | DVL1 | Predicted | Maternal |
| Blastocyst | Hypomethylated/ | chr1 | OBSCN | Predicted | Paternal |
| | Hypermethylated | | | | |
| Blastocyst | Hypermethylated | chr1 | PEX10 | Predicted | Maternal |
| Sperm | Hypomethylated/ | chr1 | PRDM16 | Predicted | Paternal |
| | Hypermethylated | | | | |
| Blastocyst | Hypomethylated | chr1 | PRDM16 | Predicted | Paternal |
| Sperm | Hypermethylated | chr1 | PTPN14 | Predicted | Maternal |
| Sperm | Hypomethylated | chr1 | TP73 | Imprinted | Maternal |
| Blastocyst | Hypomethylated | chr2 | GPR1 | Imprinted | Paternal |
| Blastocyst | Hypomethylated | chr2 | MYEOV2 | Predicted | Paternal |
| Blastocyst | Hypomethylated | chr3 | ALDHIL1 | Predicted | Maternal |
| Blastocyst | Hypermethylated | chr6 | IGF2R | Conflicting | Biallelic |
| | | | | Data | |
| Blastocyst | Hypomethylated/ | chr6 | PRIM2 | Conflicting | Biallelic |
| | Hypermethylated | | | Data | |
| Blastocyst | Hypomethylated | chr6 | ADTRP | Imprinted | Maternal |
| Sperm | Hypomethylated | chr7 | DDC | Imprinted | Isoform |
| | | | | | Dependent |
| Blastocyst | Hypomethylated | chr7 | HOXA3 | Predicted | Maternal |
| Sperm | Hypermethylated | chr7 | MAGI2 | Imprinted | Maternal |
| Blastocyst | Hypomethylated | chr7 | SLC4A2 | Predicted | Maternal |
| Sperm | Hypomethylated | chr8 | DLGAP2 | Imprinted | Paternal |
| Blastocyst | Hypomethylated | chr8 | DLGAP2 | Imprinted | Paternal |
| Blastocyst | Hypomethylated | chr8 | ZFAT | Imprinted | Paternal |
| Sperm | Hypomethylated | chr9 | EGFL7 | Predicted | Paternal |
| Sperm | Hypomethylated | chr10 | VENTX | Predicted | Maternal |
| Sperm | Hypomethylated | chr11 | ANO1 | Imprinted | Maternal |
| Sperm | Hypomethylated | chr11 | B4GALNT4 | Predicted | Maternal |
| Blastocyst | Hypomethylated | chr11 | B4GALNT4 | Predicted | Maternal |
| Sperm | Hypomethylated | chr11 | H19 | Imprinted | Maternal |
| Sperm | Hypomethylated | chr11 | IGF2;INS-IGF2 | Imprinted | Paternal |
| Sperm | Hypomethylated | chr11 | KCNQ1 | Imprinted | Maternal |
| Blastocyst | Hypomethylated/ | chr11 | KCNQ1 | Imprinted | Maternal |
| | Hypermethylated | | | | |
| Blastocyst | Hypomethylated | chr11 | NAP1L4 | Unknown | Unknown |
| Blastocyst | Hypomethylated | chr11 | NTM | Imprinted | Maternal |
| Blastocyst | Hypomethylated | chr11 | OSBPL5 | Imprinted | Maternal |
| Blastocyst | Hypomethylated | chr11 | RAB1B | Predicted | Maternal |
| Sperm | Hypomethylated | chr11 | SLC22A18AS | Provisional | Maternal |
| | | | | Data | |
| Sperm | Hypermethylated | chr11 | WT1 | Imprinted | Paternal |
| Sperm | Hypomethylated | chr12 | FBRSL1 | Predicted | Maternal |
| Blastocyst | Hypomethylated/ | chr12 | FBRSL1 | Predicted | Maternal |
| | Hypermethylated | | | | |
| Sperm | Hypermethylated | chr12 | LRP1 | Imprinted | Unknown |
| Sperm | Hypomethylated | chr14 | DLK1 | Imprinted | Paternal |
| Sperm | Hypomethylated | chr14 | RTL1 | Imprinted | Paternal |
| Sperm | Hypermethylated | chr15 | GABRG3 | Conflicting | Paternal |
| | | | | Data | |
| Blastocyst | Hypomethylated | chr15 | SNRPN | Imprinted | Paternal |
| Blastocyst | Hypomethylated | chr16 | NAA60 | Imprinted | Maternal |
| Sperm | Hypomethylated | chr16 | SOX8 | Predicted | Paternal |
| Blastocyst | Hypermethylated | chr19 | DNMT1 | Imprinted | Paternal |
| Sperm | Hypermethylated | chr19 | LILRB4 | Predicted | Maternal |
| Blastocyst | Hypermethylated | chr19 | NLRP2 | Imprinted | Maternal |
| Blastocyst | Hypermethylated | chr19 | PEG3;ZIM2; | Imprinted | Paternal |
| | | | MIMT1 | | |
| Sperm | Hypomethylated | chr19 | PPAP2C | Predicted | Maternal |
| Blastocyst | Hypermethylated | chr19 | ZNF229 | Predicted | Maternal |
| Blastocyst | Hypomethylated | chr20 | GNAS | Imprinted | Isoform |
| | | | | | Dependent |
| Sperm | Hypomethylated | chr20 | HM13 | Unknown | Unknown |
| Blastocyst | Hypomethylated | chr20 | HM13 | Unknown | Unknown |

### Conclusion

The above studies demonstrate for the first time the generational inheritance of epigenetic dysregulation from human sperm to the preimplantation embryo. The results demonstrate a mechanism for the paternal age effect from sperm to offspring, with confirmed significant susceptibility at neurodevelopmental genes associated with autism spectrum disorder, schizophrenia, and bipolar disorder. Furthermore, the genes identified in our study significantly overlapped those that were also differentially methylated in the frontal cortex and cingulate cortex of autistic brains (Nardone et al., 2014). An increase in global methylation was observed in the APA sperm epigenome; however, DMR analysis identified a comparable number of hypermethylated and hypomethylated regions. Surprisingly, among individuals, these methylation changes occurred with tight consistency at each CpG site of validated genes, seen in both the original and independent sperm cohorts. Additionally, linear regression analysis clearly illustrated significant progressive methylation alterations as paternal age increased. Interestingly, while considerable DNA methylation changes were identified between the young and APA sperm samples, differential expression of small RNAs was not observed (data not shown), suggesting that miRNA regulation is not an epigenetic mechanism affected by advancing paternal age.

There was a highly significant overlap of DMR-associated genes between the sperm and blastocyst methylomes upon paternal aging, suggesting that the same genomic regions are affected by methylation dysregulation. Methylation alterations in our datasets were not randomly distributed across the genome, but appear clustered at certain chromosomal locations. Subtelomeric regions were highly enriched for methylation alterations, particularly cytoband 19p13.3, for both sperm and blastocyst. This cytoband harbors a large number of genes (second only to cytoband 16p13.3, also significant in all three groups). Access to these genes may require a looser chromatin conformation, generating vulnerability to epigenetic alterations. Methylation in subtelomeric regions like these may escape the large-scale epigenetic reprogramming events and therefore have potential susceptibility to disruption and transmission to offspring.

As methylation alterations were not randomly distributed, it suggests that particular features of chromatin packaging in APA sperm are vulnerable to epigenetic errors, giving rise to aberrant reprogramming within the same regions in blastocysts. The inventors postulated that the DMRs identified in APA sperm colocalized with regions of retained histones. During spermiogenesis, the majority of histones are exchanged for protamines, but as many as 15% of nucleosomes are retained in regions of high CpG density and enriched at loci of developmental importance, including developmental gene promoters, imprinted loci, and genes encoding miRNAs (de Vries et al., 2013; Hammoud et al., 2009). By comparing the data provided above to those putatively bound to retained histones, previously identified by ChIP data from Hammoud et al., 2009, a statistically significant enrichment of APA sperm DMR-associated genes correlated with nucleosome retention. A comparable enrichment was observed for the directional overlapping DMRs identified in both sperm and blastocyst datasets. Thus, paternal germline methylation alterations induced by advancing age is transmitted in a chromatin context. In particular, hypomethylated DMRs were more likely to be associated with a gene, to be associated with CpG islands and flanking regions, and more likely to colocalize with mononucleosomes. Surprisingly, while the DMR-associated genes significantly colocalized with retained histones, only a handful of DMRs were situated near genes that encode miRNAs, with no significant impact to miRNA expression in APA sperm.

Both APA sperm and blastocyst DMRs exhibited significant enrichment for neurodevelopmental genes associated with autism spectrum disorder, schizophrenia, and bipolar disorder. The incidence of these neuropsychiatric conditions is known to increase progressively with increasing paternal age (de Kluiver et al., 2017), likewise DNA methylation alterations have been associated with these disorders (Rutten & Mill, 2009; Wockner et al., 2014; Wong et al., 2014). Genomic imprinting is an epigenetic phenomenon that utilizes DNA methylation to restrict gene expression to only one inherited allele. Imprinted genes play a critical role in brain development and therefore may contribute to the etiologies of neurodevelopmental conditions (Crespi, 2008). Many imprinting disorders present with autistic-like characteristics, including Beckwith-Wiedemann syndrome (chr11p15.5), Angelman syndrome, and Prader-Willi syndrome (chr15q11-13) (Crespi, 2008). Given that imprinting is established in the germline and persists through offspring by escaping widespread epigenetic reprogramming, it is reasonable to consider its involvement in mediating APA effects. In fact, advanced paternal age was associated with methylation differences in brain-expressed imprinted loci in a mouse model, with concurrent behavioral changes (Smith et al., 2013). Both APA datasets demonstrated various human-imprinted genes. DLGAP2, which has been implicated in the development of autism (Nardone et al., 2014; Rasmussen, Rasmussen, & Silahtaroglu, 2017; Soler et al., 2018), was significantly hypomethylated in both APA sperm and blastocyst groups. In addition, the cytoband classically responsible for Beckwith-Wiedemann syndrome (chr11p15.5) was significantly impacted in the APA sperm as well as the overlapping group of DMR associated genes. Hypomethylation at KCNQ1 was statistically significant in APA sperm and blastocysts and was validated in our APA sperm samples, in accordance with previously observed hypomethylation in aged human sperm (Jenkins et al., 2014).

Neurodevelopmental disorders in offspring may also manifest as epigenetic errors at genes associated with neurological development and function, including the opioid signaling pathway, in APA sperm. Opioid signaling was identified as a primary pathway affected in APA sperm and blastocysts, as well as in the combined directional overlapping DMRs, and shared by all three candidate gene lists for autism spectrum disorder, schizophrenia, and bipolar disorder. The frontal cortex in autistic brains was also significantly enriched for genes in the opioid signaling pathway and significantly overlapped our APA sperm and blastocyst DMR-associated gene lists (Nardone et al., 2014). The opioid signaling pathway in the brain is a neurotransmitter system involved in mood regulation, and abnormal brain opioid activity likely plays a role in the genesis of neurodevelopmental disorders. Increased opioid receptors are reported in autism and schizophrenia cases (Pellissier, Gandia, Laboute, Becker, & Le Merrer, 2018; Volk, Radchenkova, Walker, Sengupta, & Lewis, 2012), and there is a strong similarity between symptoms of opiate addiction and autistic symptoms (Kalat, 1978). Conversely, autistic-like symptoms have shown to be attenuated by opioid blocking in the severely mentally disabled (Sandman et al., 1983). These contrary reactions suggest that a delicate balance is required in the opioid signaling pathway, where either excess or deficiency may produce autistic-like symptoms (Pellissier et al., 2018).

CACNA1H is one gene identified in the opioid signaling pathway. It encodes CaV3.2, a T-type calcium channel abundantly expressed in the brain and implicated in neuronal function and brain development. It interacts with opioid receptors in the opioid signaling pathway to mediate analgesia (Altier & Zamponi, 2008), and missense mutations were identified in individuals with autism spectrum disorder (Splawski et al., 2006). Hypomethylation at CACNA1H was previously observed in aged human sperm compared with the same individual when he was young (Jenkins et al., 2014). Not only was aberrant DMR hypomethylation confirmed for this same gene in the APA sperm methylome and validation data, DMR hypomethylation in the preimplantation blastocyst methylome was assessed and validated in embryos derived from APA fathers. Evidently some level of methylation retention is required for CACNA1H in blastocysts, which is then lost in those derived from APA fathers. Altered CaV3.2 calcium channel activity could ultimately lead to affected neuronal function and brain development in these offspring.

DNA methylation during spermatogenesis is susceptible to errors that can be propagated to the subsequent generation. While a large proportion of the sperm ejaculate can be analyzed for epigenetic alterations, only one methylation pattern from a single sperm utilized in fertilization has potential inheritance into the blastocyst.

APA is a subtle and varying effect on the human population, and likewise, neurodevelopmental disorders exist on a spectrum due to substantial ranges of symptom severity. Not every child that is born to an APA father is autistic. As such, it would be unreasonable to expect a drastic alteration, similar to a gene mutation or knockout, in sperm DNA methylation in all APA fathers or derived APA blastocysts.

A threshold for cumulative risk exists in terms of aberrant epigenetic alterations in sperm that, if surpassed, culminates in a predisposition to disease and ultimately an observed phenotype in offspring. The above data substantiate an increasingly compromised DNA methylation profile as sperm ages, and demonstrates a generational correlation in sperm and embryo of an altered human methylation landscape associated with APA, with significant susceptibility at genes associated with neurodevelopmental disorders.

What is claimed is:

1. A method for identifying a blastocyst with increased epigenetic dysregulation, comprising:
obtaining a blastocyst; and
identifying DNA methylation errors in the blastocyst, such that epigenetic dysregulation is:
(i) correlated with a blastocyst having DNA with an overall hypomethylated shift; or
(ii) correlated with one or more autism spectrum disorder, schizophrenia, bipolar disorder, and/or opioid signaling pathway candidate genes being hypermethylated.

2. The method of claim 1, wherein the autism spectrum disorder candidate genes are selected from the group selected from ABAT, ABCA7, ADSL, ANKRD11, CACNA1C, CACNA1H, CCDC88C, CDH11, CIC, CLSTN3, CNTNAP2, CSMD1, DDX3X, DHCR7, DPP6, EHMT1, FLT1, GLIS1, GNAS, GRIN1, GRM8, HDAC6, ITGB7, KCNQ2, NRP2, NSD1, P4HA2, PACS1, PIK3R2, PXDN, SGSH, SHANK2, SHANK3, SLC38A10, TET2, TT12, ZNF804A, and UBE3B.

3. The method of claim 1, wherein the schizophrenia candidate genes are selected from the group consisting of TCF3 and ZNF804A.

4. The method of claim 1, wherein the bipolar disorder candidate genes are selected from the group consisting of COMT, DRD4, GRIN1, MBP, PRKCZ, SHANK2, TRPM2, and ZNF804A.

5. The method of claim 1, wherein the opioid signaling pathway candidate genes are selected from the group consisting of CACNA1H, GRIN1, and PRKCZ

6. A method of determining epigenetic status in a blastocyst, comprising:
determining the level of methylation in one or more neurodevelopmental disorder candidate genes in the blastocyst;
wherein the level of methylation and gene expression in the one or more neurodevelopmental disorder genes provides the epigenetic status of the blastocyst.

7. The method of claim 6, further comprising:
comparing the level of methylation in the one or more neurodevelopmental disorder genes to the level of methylation for the same one or more neurodevelopmental disorder genes under conditions defined as the blastocyst having a normal epigenetic status.

8. The method of claim 6, wherein the one or more candidate genes are selected from the group consisting of ABAT, ABCA7, ADSL, ANKRD11, CACNA1C, CACNA1H, CCDC88C, CDH11, CIC, CLSTN3, CNTNAP2, COMT, CSMD1, DDX3X, DHCR7, DPP6, DRD4, EHMT1, FLT1, GLIS1, GNAS, GRIN1, GRM8, HDAC6, ITGB7, KCNQ2, MBP, NRP2, NSD1, P4HA2, PACS1, PIK3R2, PRKCZ, PXDN, SGSH, SHANK2, SHANK3, SLC38A10, TCF3, TET2, TRPM2, TT12, ZNF804A, and UBE3B.

9. A method of determining epigenetic status in a blastocyst, comprising:
determining the global DNA methylation of the blastocyst;
wherein the level of global DNA methylation of the blastocyst provides the epigenetic status of the blastocyst, such that, a hypomethylated or hypermethylated shift in the global DNA methylation blastocyst is an abnormal epigenetic status.

10. A method for identifying a sperm cell donor with increased risk for epigenetic dysregulation, comprising:
obtaining an ejaculate sample from the sperm cell donor; and
identifying DNA methylation errors in the sperm population, such that epigenetic dysregulation is
(i) correlated with a sperm population having DNA with an overall hypomethylated shift; or
(ii) correlated with one or more autism spectrum disorder, schizophrenia, bipolar disorder, and/or opioid signaling pathway candidate genes being hypermethylated.

11. The method of claim 10, wherein the autism spectrum disorder candidate genes are selected from the group consisting of ABAT, ABCA7, ADSL, ANKRD11, CACNA1C, CACNA1H, CCDC88C, CDH11, CIC, CLSTN3, CNTNAP2, CSMD1, DDX3X, DHCR7, DPP6, EHMT1, FLT1, GLIS1, GNAS, GRIN1, GRM8, HDAC6, ITGB7, KCNQ2, NRP2, NSD1, P4HA2, PACS1, PIK3R2, PXDN, SGSH, SHANK2, SHANK3, SLC38A10, TET2, TT12, ZNF804A, and UBE3B.

12. The method of claim 1, wherein the schizophrenia candidate genes are selected from the group consisting of TCF3 and ZNF804A.

13. The method of claim 1, wherein the bipolar disorder candidate genes are selected from the group consisting of COMT, DRD4, GRIN1, MBP, PRKCZ, SHANK2, TRPM2, and ZNF804A.

14. The method of claim 10, wherein the opioid signaling pathway candidate genes are selected from the group consisting of CACNA1H, GRIN1, and PRKCZ

15. A method of determining epigenetic status in a sperm population, comprising:
determining the degree of methylation in one or more neurodevelopmental disorder candidate genes in the sperm population;
wherein, the level of methylation in the one or more neurodevelopmental disorder genes provides the epigenetic status of the sperm sample.

16. The method of claim 15, further comprising:
comparing the level of methylation in the one or more neurodevelopmental disorder genes to the level of methylation for the same one or more neurodevelopmental disorder genes under conditions defined as the sperm population having a normal epigenetic status.

17. The method of claim 15, wherein the one or more candidate genes are selected from the group consisting of ABAT, ABCA7, ADSL, ANKRD11, CACNA1C, CACNA1H, CCDC88C, CDH11, CIC, CLSTN3, CNTNAP2, COMT, CSMD1, DDX3X, DHCR7, DPP6, DRD4, EHMT1, FLT1, GLIS1, GNAS, GRIN1, GRM8, HDAC6, ITGB7, KCNQ2, MBP, NRP2, NSD1, P4HA2, PACS1, PIK3R2, PRKCZ, PXDN, SGSH, SHANK2, SHANK3, SLC38A10, TCF3, TET2, TRPM2, TT12, ZNF804A, and UBE3B.

18. A method of determining epigenetic status in a sperm population, comprising:
determining the degree of global DNA methylation of the sperm population;
wherein, the degree of global DNA methylation of the sperm population provides the epigenetic status of the sperm population, such that, a hypomethylated or hypermethylated shift in the global DNA methylation sperm population is an abnormal epigenetic status.

19. A kit for testing the epigenetic status of a sperm population, comprising:
means for determining degree of methylation of the sperm population in one or more target genes in the blastocyst.

20. The kit of claim 19, wherein:
the one or more target genes are selected from the group consisting of ABAT, ABCA7, ADSL, ANKRD11, CACNA1C, CACNA1H, CCDC88C, CDH11, CIC, CLSTN3, CNTNAP2, COMT, CSMD1, DDX3X, DHCR7, DPP6, DRD4, EHMT1, FLT1, GLIS1, GNAS, GRIN1, GRM8, HDAC6, ITGB7, KCNQ2, MBP, NRP2, NSD1, P4HA2, PACS1, PIK3R2, PRKCZ, PXDN, SGSH, SHANK2, SHANK3, SLC38A10, TCF3, TET2, TRPM2, TT12, ZNF804A, and UBE3B.
